(19) United States
(12) Patent Application Publication
(10) Pub. No.: US 2013/0338690 A1
(43) Pub. Date: Dec. 19, 2013

(54) DEVICE AND METHOD FOR REMOVING UNWANTED MATERIAL IN A VASCULAR CONDUIT

(71) Applicant: Gadal Consulting, LLC, (US)

(72) Inventors: Florin Gadalean, Altamonte Springs, FL (US); Vladimir Grigorov, Kempton Park (ZA)

(21) Appl. No.: 13/788,232

(22) Filed: Mar. 7, 2013

Related U.S. Application Data
(63) Continuation-in-part of application No. 13/756,326, filed on Jan. 31, 2013.
(60) Provisional application No. 61/689,854, filed on Jun. 15, 2012.

(51) Int. Cl. A61B 17/3207

(52) U.S. Cl. CPC A61B 17/32075
USPC 606/159

(57) ABSTRACT

In one embodiment, a device for extracting unwanted material in a vascular conduit of a subject is provided, including an elongate member having a proximal end and a distal end, for introduction into a vascular conduit of a subject. A material engaging structure is disposed on the elongate member, and is configured to expand to contact an inner surface of the vascular conduit to fragment and/or detach unwanted material in the conduit. The material engaging structure includes a contacting structure disposed on and rotatable about the elongate member, at least a first stripping member, having a first end and a second end, wherein the first end is associated with the elongate member and the second end is associated with the contacting structure, and a netting structure configured to collect and remove the unwanted material from the vascular conduit.

### CROSS REFERENCE RELATED APPLICATIONS

This application is a continuation in part of U.S. Ser. No. 13/756, 326 filed Jan. 31, 2013, and claims priority to U. S. Provisional Application No. 61/689,854 filed Jun. 15, 2012 both of which are incorporated herein in their entirety.

### FIELD OF INVENTION

The invention relates to a device for mechanically removing a fibrin sheath or pseudo-intimal material from a vasculature of a subject. The device is used percutaneously, to obviate the need for invasive surgical procedures.

### BACKGROUND

Patients undergoing hemodialysis over an extended period of time encounter various problems during their duration of treatment. One significant problem for these patients is stenosis of their dialysis access grafts. Dialysis access grafts are synthetic grafts placed under the skin which become an artificial vein that can be used repeatedly for needle placement and blood access during hemodialysis. Stenosis is the abnormal narrowing of a blood vessel or other tubular organ or structure. Stenosis of the vasculature (including synthetic access grafts) often occurs in hemodialysis patients. This condition contributes significantly to patient morbidity and hospitalization.

The prior art has provided techniques and various instruments to attempt to break up clots and/or other obstructing materials in the vasculature and synthetic grafts to avoid the aforementioned problems. Although surgery has been the traditional method of management for thrombosed access grafts and fistulae, surgery is often invasive and carries with it many potential complications. There are various existing techniques employed to reestablish blood flow in an occluded blood vessel. One common surgical technique, an embolectomy, involves incising a blood vessel and introducing a balloon-tipped device (e.g., the Fogarty catheter) to the location of the occlusion. The balloon is then inflated at a point beyond the clot and used to translate the obstructing material back to the point of incision. The obstructing material is then removed by the surgeon. While such surgical techniques have been useful, exposing a patient to surgery may be traumatic and best avoided whenever possible. Additionally, certain risks of these techniques include possible damage to the interior lining of the vessel as the balloon tipped device is being withdrawn.

Percutaneous methods are also utilized to reestablish blood flow in the vasculature of a patient. A common percutaneous technique includes a balloon angioplasty where a balloon-tipped catheter is introduced into a blood vessel, typically through an introducing catheter. The balloon-tipped catheter is then advanced to the point of the occlusion and inflated in order to dilate the stenosis. These methods and techniques, however, fail to remove the debris from the vasculature once the clot/obstruction is destroyed.

### SUMMARY

In one embodiment herein, a device for extracting unwanted material in a vascular conduit of a subject is provided. The device includes an elongate member including a proximal end and a distal end, the distal end for introduction into a vascular conduit of a subject, a material engaging structure disposed on the elongate member, the material engaging structure configured to expand to contact an inner surface of the vascular conduit to fragment and/or detach unwanted material in the conduit. The material engaging structure includes a contacting structure disposed on and rotatable about the elongate member, at least a first stripping member, the first stripping member including a first end and a second end, wherein the first end is associated with the elongate member and the second end is associated with the contacting structure, and a netting structure is disposed between the first stripping member and the elongate member from the first end to the second end, the netting structure configured to collect and remove the unwanted material from the vascular conduit. The material engaging structure further including a collection structure removably affixed to the distal end of the elongate member, wherein the collection structure is configured to collect and remove the unwanted material from the vascular conduit, wherein movement of the contacting structure toward the distal end of the member is configured to expand the collection structure and the material engaging structure to the shape and diameter of the vascular conduit, wherein rotation of the device when the in the vascular conduit fragments and/or detaches the unwanted material in the conduit, and wherein movement of the contacting structure toward the proximal end of the member collapses the material engaging structure and the collection structure such that the unwanted material can be collected and removed from the conduit.

In another embodiment, a method for extracting a pseudo-intimal and/or unwanted material in a vascular conduit of a subject is provided. The method includes inserting a device including an elongate member, the elongate member including a material engaging structure in a compressed state, the material engaging structure including at least a first stripping member and a netting structure disposed between the first stripping member and the elongate member, wherein the device is slidably received within an outer sheath assembly, into a vascular conduit of a subject. The method further includes advancing the material engaging structure within the vascular conduit to a location proximal to the pseudo-intimal and/or unwanted material in the vascular conduit to be extracted, deploying the material engaging structure within the vascular conduit such that the material engaging structure expands to conform to the shape and/or diameter of the vascular conduit, rotating the device such that the material engaging structure engages and/or detaches the pseudo-intimal material/and or unwanted material from the vascular conduit, and retaining the pseudo-intimal and/or unwanted material in the netting structure and/or a collection structure disposed at a distal end of the device. The method further includes compressing the material engaging structure, and removing the device including the pseudo-intimal and/or unwanted material from the vascular conduit.

In still another embodiment, a device for extracting unwanted material in a vascular conduit of a subject is provided. The device includes an elongate member including a proximal end and a distal end, the distal end for introduction into a vascular conduit of a subject, a material engaging structure disposed on the elongate member, the material engaging structure configured to expand to contact an inner surface of the vascular conduit to fragment and/or detach unwanted material in the conduit. The material engaging structure includes a contacting structure disposed on and rotatable about the member, at least a first stripping member and a second stripping member, the first and second stripping members each including a first end and a second end. The second ends of the first and second stripping members are associated with the contacting structure, and the first ends are associated with the elongate member. A netting structure is disposed between each of the first stripping member and second stripping member and the elongate member from the first end to the second end. In some instances the netting structure extends from the first end to a midpoint on the stripping member. The netting structure is configured to collect and remove the unwanted material from the vascular conduit. A collection structure is removably affixed to the distal end of the elongate member, wherein the collection structure is configured to collect and remove the unwanted material from the vascular conduit, wherein movement of the contacting structure toward the distal end of the member is configured to expand the collection structure and the material engaging structure to the shape and/or diameter of the vascular conduit, and wherein rotation of the device fragments and/or detaches the unwanted material in the conduit, whereby movement of the contacting structure toward the proximal end of the member collapses the material engaging structure and the collection structure such that the unwanted material can be collected and removed from the conduit.

### BRIEF DESCRIPTION OF THE DRAWINGS

FIG. 1 provides a perspective view of an embodiment of the device.

FIG. 2 provides a side view of an embodiment of the device in a compressed state.

FIG. 3 provides a side view of an embodiment of the device in an expanded state.

FIG. 4 provides a perspective view of another embodiment of the device.

### DETAILED DESCRIPTION

The inventor has discovered a device and method which in one embodiment engages and removes a pseudo-intimal material, and foreign bodies, particles or fragments from within a vascular conduit. These fragments, foreign bodies, particles or other debris can obstruct a vessel and cause damaging effects to a patient if not removed from the vasculature. There are several devices used for retrieval and removal of foreign bodies which include, for example, loop-like structures for insertion into the vasculature containing the foreign body. However, these devices are either highly complex and include difficulties in manufacture, and furthermore are difficult to operate. Moreover, the currently available devices are insufficient in retaining the foreign bodies or debris, and the fail to appreciate the need for removal of the pseudo-intimal material which builds up within the vasculature or synthetic grafts of a patient.

The subject invention provides, in some embodiments, a device and a method to remove the pseudointimal material and fibrin sheath within the vascular system, and in particular, within synthetic vascular grafts, in some embodiments. The invention overcomes deficiencies in the prior art, some of which have included the inability of a device to: 1) expand to conform to the inner lumen dimensions and shape; 2) apply a radial pressure so that the device automatically expands and engages the fibrin sheath; 3) use a minimal number and size of components so that the catheter can be deployed through a small introducer sheath; and 4) place the device through the arterial anastmosis without causing damage to the artery while being capable of removing pseudointimal hyperplasia/fibrin sheath (i.e., pseudointimal material) with precision from critical and specific areas in the vasculature. The subject invention provides advantages over the prior art including decreasing procedure time, decreasing cost and risk, allowing the use of a smaller sheath size, and automatically accommodating for differences in the inner diameter of the vessel.

The subject invention is directed to devices and methods useful in removing clots, foreign bodies and pseudo-intimal material from vessels. Various embodiments are disclosed for the effective removal of these clots, foreign bodies and pseudo-intimal materials. It is contemplated that the present invention may be used in all vasculature.

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains. It is to be noted that all ranges disclosed within this specification are inclusive and are independently combinable.

Other objects, features, and characteristics of the present invention as well as the methods of operation and functions of the related elements of structure and the combination of parts and the economies of manufacture, will become more apparent upon consideration of the following description and the appended claims with reference to the accompanying drawings, all of which form a part of this specification, wherein like reference numerals designate corresponding parts in the various figures.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

### DEFINITIONS

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), most preferably a human.

As used herein, the terms “foreign material”, “foreign body”, “foreign matter” and “debris” are used interchangeably. As used herein, these terms may refer to any type of unwanted material in the vasculature including but not limited to fragments of catheters, pace-maker electrodes, guide wires, erroneously placed embolic material such as thrombogenic coils, and other such foreign material as known in the art. Unwanted material may also refer to thrombotic clot fragments, thrombotic material, pseudo-intimal material, fibrin sheath, or any combination thereof.

The term “intertwined” as used herein refers to an overlap of the members of the device, whether the members are in contact or not in contact with one another.

As used herein, the term “pseudo-intima” and “pseudo-intimal material” refers to the material that builds up in the inner portion of the vasculature of a patient, particularly but not necessarily in hemodialysis patients.

A “fibrin sheath” as discussed herein, refers to in one example, a tubular scar that is a potential complication of long-term catheterization, in which the catheter becomes encased in a fibrotic sheath, which may harbor bacteria and make it difficult to withdraw blood from the line.

The term “proximal” as used herein refers to a location that is near the end of the device closest to the end at which the device is held by a user during its use.

The term “distal” as used herein refers to a location that is near the end of the device nearest the entry point for the device in the subject.

In one embodiment herein, a device for extracting unwanted material in a vascular conduit of a subject is provided. The device includes an elongate member including a proximal end and a distal end, the distal end for introduction into a vascular conduit of a subject, a material engaging structure disposed on the elongate member, the material engaging structure configured to expand to contact an inner surface of the vascular conduit to fragment and/or detach unwanted material in the conduit. The material engaging structure includes a contacting structure disposed on and rotatable about the elongate member, at least a first stripping member, the first stripping member including a first end and a second end, wherein the first end is associated with the elongate member and the second end is associated with the contacting structure, and a netting structure is disposed between the first stripping member and the elongate member from the first end to the second end, the netting structure configured to collect and remove the unwanted material from the vascular conduit.

The material engaging structure further including a collection structure removably affixed to the distal end of the elongate member, wherein the collection structure is configured to collect and remove the unwanted material from the vascular conduit, wherein movement of the contacting structure toward the distal end of the member is configured to expand the collection structure and the material engaging structure to the shape and diameter of the vascular conduit, wherein rotation of the device when the in the vascular conduit fragments and/or detaches the unwanted material in the conduit, and wherein movement of the contacting structure toward the proximal end of the member collapses the material engaging structure and the collection structure such that the unwanted material can be collected and removed from the conduit.

In a further embodiment, the material engaging structure comprises a compressed state and an expanded state, wherein in a compressed state, the material engaging structure may be slidably received within an outer sheath assembly for delivery of the material engaging structure to a location proximal to the unwanted material in the vascular conduit, and in an expanded state, the material engaging structure is in contact with an inner surface of the vascular conduit. In another embodiment, the collection structure and/or the netting structure comprises a mesh material to capture unwanted material from the vascular conduit.

In one embodiment, the unwanted material comprises thrombotic material or pseudo-intimal material, or a combination thereof.

In another embodiment, the device is coupled to a rotator unit, wherein the rotator unit rotates the material engaging structure to engage and/or remove the unwanted material from the vascular conduit. In yet another embodiment, the device is coupled to a motor to rotate the device.

In a further embodiment, the at least a first stripping member is comprised of a wire wherein at least a portion of the wire is flat or round. In other embodiments, two or more stripping members may be used, such stripping members may be intertwined, in one embodiment. In a further embodiment, the wire or wires are formed in a straight or helical configuration. In a further embodiment, the stripping member includes a cutting edge.

In another embodiment, the elongate member comprises a passageway there through such that a fluid can be introduced into the conduit through the elongate member to remove debris from the conduit. Other materials may also be introduced through the passageway in the elongate member, such as a guide wire, and other such tools used for procedures.

In another embodiment, a locking mechanism is provided such that the material engaging structure can be locked in an expanded or a compressed state. The locking mechanism may be coupled to the contacting structure, to lock the contacting structure in a particular position, therefore locking the material engaging structure in an expanded or compressed state. The locking mechanism may take other embodiments and may be associated with other parts of the device.

In yet another embodiment, a method for extracting a pseudo-intimal and/or unwanted material in a vascular conduit of a subject is provided. The method includes inserting a device including an elongate member, the elongate member including a material engaging structure in a compressed state, the material engaging structure including at least a first stripping member and a netting structure disposed between the first stripping member and the elongate member, wherein the device is slidably received within an outer sheath assembly, into a vascular conduit of a subject. The method further includes advancing the material engaging structure within the vascular conduit to a location proximal to the pseudo-intimal and/or unwanted material in the vascular conduit to be extracted, deploying the material engaging structure within the vascular conduit such that the material engaging structure expands to conform to the shape and/or diameter of the vascular conduit, rotating the device such that the material engaging structure engages and/or detaches the pseudo-intimal material/and or unwanted material from the vascular conduit, and retaining the pseudo-intimal and/or unwanted material in the netting structure and/or a collection structure disposed at a distal end of the device. The method further includes compressing the material engaging structure, and removing the device including the pseudo-intimal and/or unwanted material from the vascular conduit.

In still another embodiment, the method is provided wherein a contacting structure is disposed on the elongate member, and wherein when the contacting structure is shifted toward a distal end of the device the material engaging structure is expanded. In another embodiment, the method is provided wherein the contacting structure is shifted toward a proximal end of the device to compress the material engaging structure.

In a further embodiment, the method is provided wherein the device is coupled to a rotator unit, wherein the rotator unit rotates the material engaging structure to engage and/or detach the pseudo-intimal and/or unwanted material from the vascular conduit.

In still a further embodiment, the method is provided wherein the first stripping member comprises a first end and a second end, wherein the first end is associated with the contacting structure, wherein the contacting structure is slidably engaged with the elongate member, and wherein the second end is associated with the distal end of the device. In a further embodiment, the first stripping member includes a netting structure disposed along a portion of the first stripping member between the first stripping member and the elongate member, the netting structure extending from the second end of the stripping member down a portion of the length of the stripping member, wherein the netting structure engages and removes the pseudo-intimal and/or unwanted material from the vascular conduit. In yet a further embodiment, the unwanted material comprises thrombotic material or a foreign material, or a combination thereof.

In another embodiment, the elongate member is hollow, and a fluid can be introduced into the conduit through the elongate member to remove debris from the conduit. Other materials can also be introduced into the vascular conduit through the elongate member, such as a wire used to perform a procedure in the vascular conduit or another type of conduit, or other materials or substances can be introduced through the elongate member.

In a further embodiment, the method is provided wherein the device comprises a locking mechanism such that the material engaging structure can be locked in an expanded or a compressed state.

In still a further embodiment, a motor is coupled to the device to rotate and spin the device, enabling the unwanted material in the vascular conduit to become trapped by the netting structure and/or a collection structure at the distal end of the elongate member. The motor provides easibility of use of the device, particularly wherein the device includes one stripping member. The motor can be powered on and off with the simple movement of a switch associated with the device.

The collection structure may be disposed or removably affixed to the distal end of the elongate member. The collection structure is configured to collect and remove the unwanted material from the vascular conduit, wherein movement of the contacting structure toward the distal end of the member expands the collection structure to the shape and diameter of the vascular conduit and movement of the contacting structure toward the proximal end of the elongate member compresses the collection structure. The collection structure collects unwanted material to be removed from the inner portion of the vascular conduit. The collection structure can include a mesh-type material, wherein debris for removal is trapped within the collection structure when it is in an expanded state inside the conduit. When the collection structure is collapsed prior to removal of the device from the conduit, the debris/unwanted material trapped within the collection structure is removed from the conduit.

In still another embodiment, a device for extracting unwanted material in a vascular conduit of a subject is provided. The device includes an elongate member including a proximal end and a distal end, the distal end for introduction into a vascular conduit of a subject, a material engaging structure disposed on the elongate member, the material engaging structure configured to expand to contact an inner surface of the vascular conduit to fragment and/or detach unwanted material in the conduit. The material engaging structure includes a contacting structure disposed on and rotatable about the member, at least a first stripping member and a second stripping member, the first and second stripping members each including a first end and a second end. The second ends of the first and second stripping members are associated with the contacting structure, and the first ends are associated with the elongate member. A netting structure is disposed between each of the first stripping member and second stripping member and the elongate member from the first end to the second end. In some instances the netting structure extends from the first end to a midpoint on the stripping member. The netting structure is configured to collect and remove the unwanted material from the vascular conduit.

A collection structure is removably affixed to the distal end of the elongate member, wherein the collection structure is configured to collect and remove the unwanted material from the vascular conduit, wherein movement of the contacting structure toward the distal end of the member is configured to expand the collection structure and the material engaging structure to the shape and/or diameter of the vascular conduit, and wherein rotation of the device fragments and/or detaches the unwanted material in the conduit, whereby movement of the contacting structure toward the proximal end of the member collapses the material engaging structure and the collection structure such that the unwanted material can be collected and removed from the conduit.

A vascular sheath can be used as a port of entry for the device in the vasculature, in one embodiment. The device can be inserted into and removed from the vasculature thorough the vascular sheath.

In an expanded state, the material engaging structure is provided for contacting the inner surface of the vascular conduit. The material engaging structure is deployed from the outer sheath assembly before it is positioned in the expanded state. The outer sheath assembly can range in diameter between 5-9 French, for example. The outer sheath assembly includes a hemostasis hub connected thereto. In order to minimize trauma to vascular grafts, the smallest possible outer sheath is used. The outer sheath assembly provides ease in placement of the device into the vasculature prior to deployment and expansion of the material engaging structure proximal to the unwanted material in the conduit. In one particular embodiment, the outer sheath assembly includes a catheter.

The hemostasis hub includes a silicone seal and a cap to prevent fluids, such as blood, from contacting the user, in one embodiment. In another embodiment, a side arm may be provided for flushing out the lumen before use and/or for injecting contrast during the procedure.

The material engaging structure portions may spin in excess of approximately 500 rpm in one embodiment. In some embodiments the stripping members may be formed of nickel titanium (nitinol). In other embodiments, stainless steel may be used.

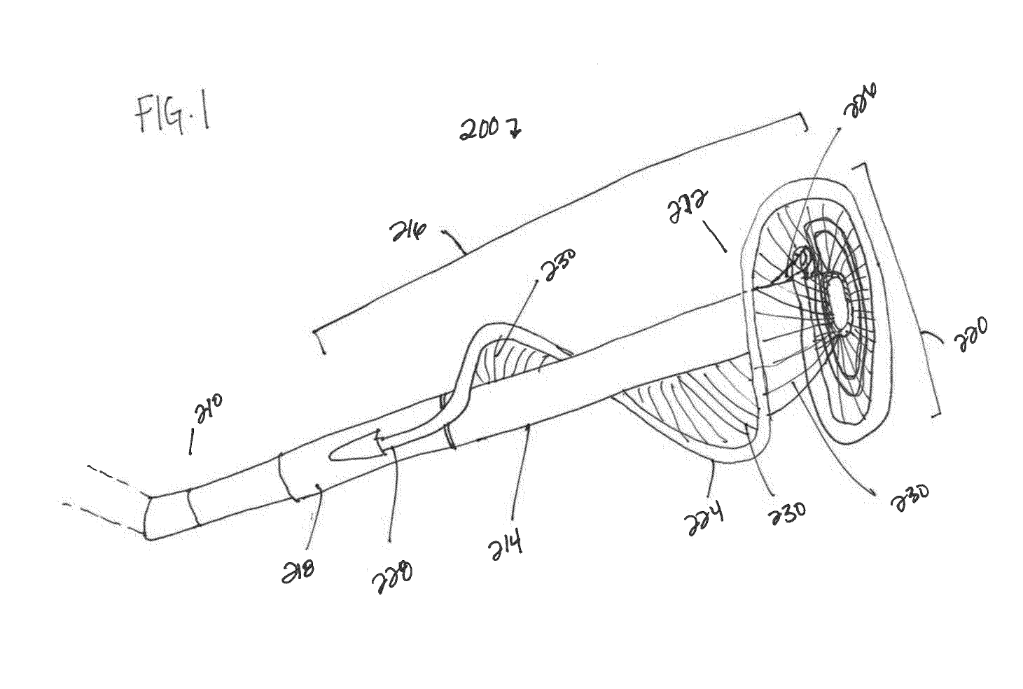

Turning to the drawings, FIG. 1 provides a perspective view of an embodiment of the device 200, wherein a first stripping member 224 having a proximal end 228 and a distal end 226 is provided. An elongate member 214 is shown with the proximal end 210 and the distal end 212. The material engaging structure portion 216 includes the first stripping member 224 and the collection structure 220. The netting structure 230 is disposed between the first stripping member 224 and the elongate member 214. The contacting structure 218 can be seen in FIG. 1.

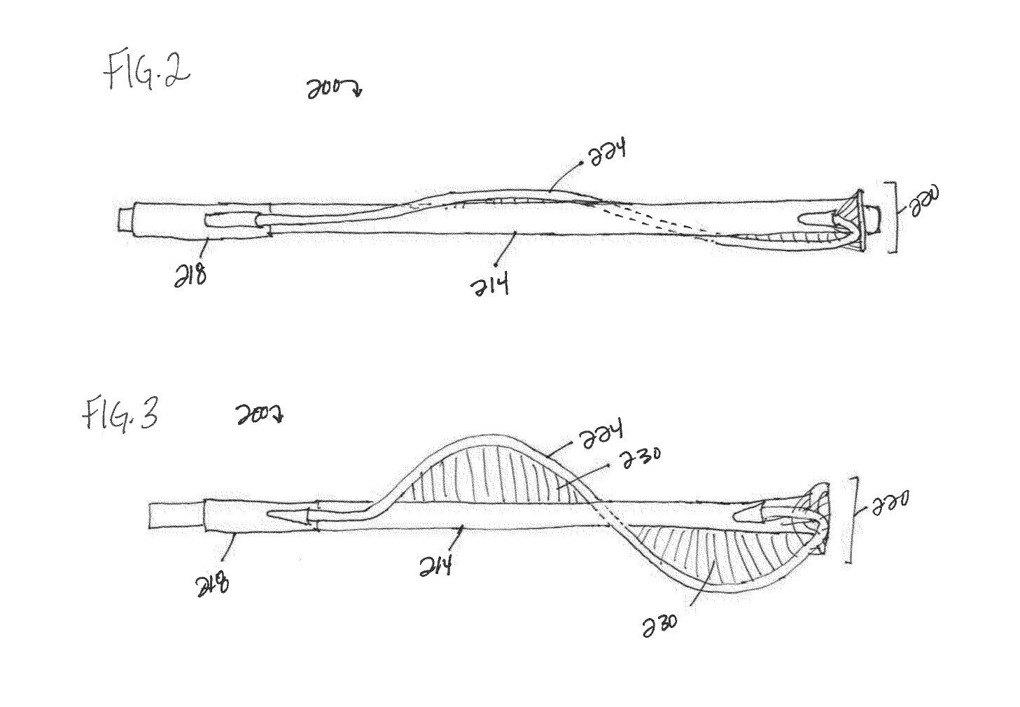

FIGS. 2 and 3 provide a side view of the embodiment of the device 200 as shown in FIG. 1, wherein the device 200 is shown in a compressed state (FIG. 2) with the contacting device 218 at the proximal end of the elongate member 214, and in an expanded state (FIG. 3) with the contacting member 218 shifted toward the distal end of the elongate member 214.

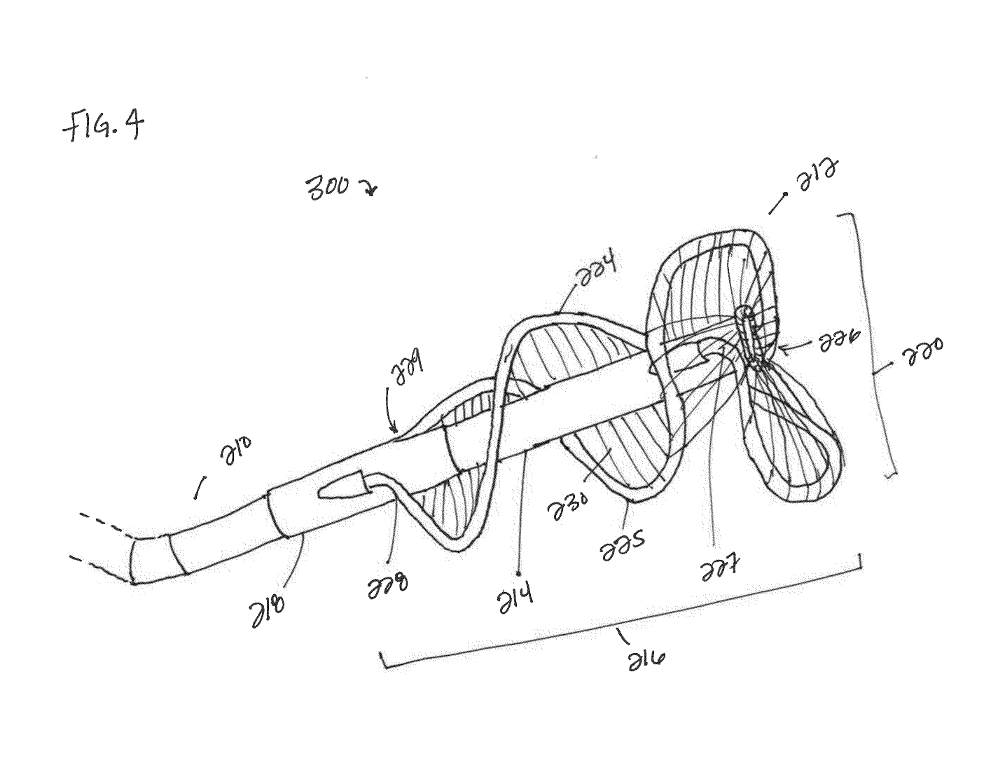

FIG. 4 provides a perspective view of another embodiment of the device 300. This embodiment 300 includes both a first striping member 224 and a second stripping member 225. The first stripping member 224 having a proximal end 228 and a distal end 226, and the second stripping member having a proximal end 229 and a distal end 227. The material engaging structure 216 includes the stripping members 24, 225 and the collection structure 220. The contacting structure 218 is also shown in FIG. 4, along with the netting structure 230 disposed between each of the stripping members 224, 225 and the elongate member 214.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A device for extracting unwanted material in a vascular conduit of a subject, comprising:
an elongate member comprising a proximal end and a distal end, the distal end for introduction into a vascular conduit of a subject;
a material engaging structure disposed on the elongate member, the material engaging structure configured to expand to contact an inner surface of the vascular conduit to fragment and/or detach unwanted material in the conduit, the material engaging structure comprising
a contacting structure disposed on and rotatable about the elongate member;
at least a first stripping member, the first stripping member comprising a first end and a second end, wherein the first end is associated with the elongate member and the second end is associated with the contacting structure, and a netting structure is disposed between the first stripping member and the elongate member from said first end to said second end, said netting structure configured to collect and remove the unwanted material from the vascular conduit; and
a collection structure removably affixed to the distal end of the elongate member, wherein the collection structure is configured to collect and remove the unwanted material from the vascular conduit;
wherein movement of the contacting structure toward the distal end of the member is configured to expand the collection structure and the material engaging structure to the shape and diameter of the vascular conduit, wherein rotation of the device when the in the vascular conduit fragments and/or detaches the unwanted material in the conduit, and wherein movement of the contacting structure toward the proximal end of the member collapses the material engaging structure and the collection structure such that the unwanted material can be collected and removed from the conduit.

2. The device of claim 1, wherein said material engaging structure comprises a compressed state and an expanded state, wherein in a compressed state, the material engaging structure may be slidably received within an outer sheath assembly for delivery of the material engaging structure to a location proximal to the unwanted material in the vascular conduit, and in an expanded state, the material engaging structure is in contact with an inner surface of the vascular conduit.

3. The device of claim 1, wherein the collection structure and/or the netting structure comprises a mesh material to capture unwanted material from the vascular conduit.

4. The device of claim 1, wherein the unwanted material comprises thrombotic material or pseudo-intimal material, or a combination thereof.

5. The device of claim 1, wherein the device is coupled to a rotator unit, wherein the rotator unit rotates the material engaging structure to engage and/or remove the unwanted material from the vascular conduit.

6. The device of claim 1, wherein the device is coupled to a motor to rotate the device.

7. The device of claim 1, wherein the at least a first stripping member is comprised of a wire wherein at least a portion of the wire is flat or round.

8. The device of claim 7, wherein said wire is formed in a straight or helical configuration.

9. The device of claim 1, wherein the stripping member comprises a cutting edge.

10. The device of claim 1, wherein the elongate member comprises a passageway there through such that a fluid can be introduced into the conduit through the elongate member to remove debris from the conduit.

11. The device of claim 1, further comprising a locking mechanism such that the material engaging structure can be locked in an expanded or a compressed state.

12. A method for extracting a pseudo-intimal and/or unwanted material in a vascular conduit of a subject, said method comprising:
inserting a device comprising an elongate member comprising a material engaging structure in a compressed state, said material engaging structure comprising at least a first stripping member and a netting structure disposed between the first stripping member and the elongate member, wherein said device is slidably received within an outer sheath assembly, into a vascular conduit of a subject;
advancing the material engaging structure within the vascular conduit to a location proximal to the pseudo-intimal and/or unwanted material in the vascular conduit to be extracted;
deploying the material engaging structure within the vascular conduit such that the material engaging structure expands to conform to the shape and/or diameter of the vascular conduit;
rotating the device such that the material engaging structure engages and/or detaches the pseudo-intimal material/and or unwanted material from the vascular conduit;
retaining the pseudo-intimal and/or unwanted material in the netting structure and/or a collection structure disposed at a distal end of the device;
compressing the material engaging structure; and
removing the device comprising the pseudo-intimal and/or unwanted material from the vascular conduit.

13. The method of claim 12, wherein a contacting structure is disposed on the elongate member, and wherein when the contacting structure is shifted toward a distal end of the device the material engaging structure is expanded.

14. The method of claim 13, wherein the contacting structure is shifted toward a proximal end of the device to compress the material engaging structure.

15. The method of claim 12, wherein the device is coupled to a rotator unit, wherein the rotator unit rotates the material engaging structure to engage and/or detach the pseudo-intimal and/or unwanted material from the vascular conduit.

16. The method of claim 13, wherein the first stripping member comprises a first end and a second end, wherein said first end is associated with the contacting structure, wherein said contacting structure is slidably engaged with the elongate member, and wherein the second end is associated with the distal end of the device.

17. The method of claim 13, wherein the first stripping member comprises a netting structure disposed along a portion of the first stripping member between the first stripping member and the elongate member, the netting structure extending from the second end of the stripping member down a portion of the length of the stripping member, wherein said netting structure engages and removes the pseudo-intimal and/or unwanted material from the vascular conduit.

18. The method of claim 12, wherein the unwanted material comprises thrombotic material or a foreign material, or a combination thereof.

19. The method of claim 12, wherein the elongate member is hollow, and a fluid can be introduced into the conduit through the elongate member to remove debris from the conduit.

20. The method of claim 12, wherein the device comprises a locking mechanism such that the material engaging structure can be locked in an expanded or a compressed state.

21. The method of claim 12, wherein a motor is coupled to the device to rotate the device.

22. A device for extracting unwanted material in a vascular conduit of a subject, comprising:
an elongate member comprising a proximal end and a distal end, the distal end for introduction into a vascular conduit of a subject;
a material engaging structure disposed on the elongate member, the material engaging structure configured to expand to contact an inner surface of the vascular conduit to fragment and/or detach unwanted material in the conduit, the material engaging structure comprising
a contacting structure disposed on and rotatable about the member;
at least a first stripping member and a second stripping member, the first and second stripping members each comprising a first end and a second end, wherein the second ends of the first and second stripping members are associated with the contacting structure, and the first ends are associated with the elongate member, and a netting structure is disposed between each of the first stripping member and second stripping member and the elongate member and between said first end and said second end, said netting structure configured to collect and remove the unwanted material from the vascular conduit; and
a collection structure removably affixed to the distal end of the elongate member, wherein the collection structure is configured to collect and remove the unwanted material from the vascular conduit;
wherein movement of the contacting structure toward the distal end of the member is configured to expand the collection structure and the material engaging structure to the shape and/or diameter of the vascular conduit, and wherein rotation of the device fragments and/or detaches the unwanted material in the conduit, whereby movement of the contacting structure toward the proximal end of the member collapses the material engaging structure and the collection structure such that the unwanted material can be collected and removed from the conduit.
